(19) United States
(12) Patent Application Publication
(10) Pub. No.: US 2005/0143933 A1
(43) Pub. Date: Jun. 30, 2005

(54) Analyzing and correcting biological assay data using a signal allocation model

(76) Inventor: James Minor, Los Altos, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/167,119

(22) Filed: Jun. 10, 2002

Related U.S. Application Data
(60) Provisional application No. 60/375,251, filed on Apr. 23, 2002.

(51) Int. Cl. G06F 19/00; G01N 33/48; G01N 33/50

(52) U.S. Cl. 702/20

(57) ABSTRACT

Data from a biological assay are analyzed and corrected to deconvolve and estimate the expression of a target material using the measured signals from a target probe and on or more homologous probes. The expressions of target and non-target material in a biological sample are allocated to the measured signals of multiple probes. The SIAM is used to correct the biological assay data to obtain more accurate results for the true expression.

### CROSS REFERENCE TO RELATED APPLICATIONS

This application claims the benefit under 35 U.S.C. § 119(e) of U.S. Provisional Application Ser. No. 60/375,251, filed Apr. 23, 2002, which is herein incorporated in its entirety by reference.

### BACKGROUND

1. Field of the Invention

The present invention relates generally to techniques for analyzing biological assay data having a plurality of signals. In particular, the invention is applied to deconvolve and estimate the expression of a target material using the measured signals from a target probe and one or more homologous probes.

2. Background of the Invention

Advances in microarray technology have enabled researchers to monitor a large numbers of genes and other biological materials in parallel on a single microarray chip. Array technology is used, for example, to follow the changes in the expression levels of multiple genes, to identify distinctive expression patterns characteristic of physiological and pathological states, and to screen for changes in the response to a particular therapeutic treatment. In this context, the expression of a material is a measure of its abundance in a sample. Using the biological assay data obtained from such microarrays and other similar research test equipment, researchers diagnose diseases, develop medical treatments, understand biological phenomena, and perform other tasks relating to the analysis of the data.

However, the conversion of useful results from this raw data is restricted by physical limitations and data analysis techniques. For example, the data obtained from a microarray experiment include signals that are related to the amount of bonding of a target material to probes at various locations on the microarray. These signals, however, may be affected by more than just the bonding of each material to its associated probe. In a genetic experiment for example, bonding due to cross-hybridization of nonspecific species and other background “noise” effects may also contribute to the signals measured by each probe. Because of these noise effects, the assay data is often unusable where the signal intensities are low relative to the noise and/or cross-hybridization or other similar effects. In such a case, the noise outweighs the useful biological information in the data, and existing methods fail to provide an effective means of extracting the useful biological information from such assay data.

One type of assay is a microarray that includes different probes at various locations or spots on the array (typically in a grid pattern). Each probe is formed of oligonucleotides of a particular sequence immobilized at a location on the microarray. The probes are placed in contact with a sample containing target material, which includes oligonucleotide sequences that can bond with the immobilized sequences on the array. The target material is further bonded to a phosphorescent, fluorescent, or other energy-emitting material. Once the target material is placed in contact with the probes, it is allowed to bond with the probes on the array. The binding of sequences is driven by their chemical affinity and concentrations. In addition, the sample usually has non-target material, which may also bind to the probes.

After the target and non target material is allowed to bond to various probes on the microarray, the array is photo-scanned to measure the intensity of the energy produced by the energy-emitting material bonded at each location. The light intensity at a location is monotonically related to the bonding of target material at the location, which in turn corresponds to the expression of the particular target genetic sequence. (Typically, the intensity of a probe's signal is computed from the mean of the pixel intensities at the probe's location on the array.) This measured light or other energy intensity is the probe's signal.

One specific microarray commonly used in such an assay is the glass spot array, such as the GENECHIP® brand arrays made by Affymetrix, Inc. of Santa Clara, Calif., described in U.S. Pat. No. 5,968,740. Oligo-microarrays such as the GENECHIP® (or “Affy”) arrays are designed with multiple probe-pairs for detecting different genetic subsequences specific to one or more genes. FIG. 1 schematically illustrates a side-view of a portion of such a microarray 110. In this microarray, a probe-pair consists of two probes, a “perfect match” (PM) probe 120 and an adjacent “mismatch” (MM) probe 130. A PM probe 120 comprises a number of oligonucleotide that correspond to a target material 140 (e.g., nucleic subsequence of the gene), while the MM probe 130 contains a perturbation relative to the perfect match's sequence. Typically, the mismatch sequence is identical to the perfect match sequence except that one nucleic acid component is altered in the middle of the sequence.

The measured signal from each probe in the probe-pair is proportional or monotonically correlated to the amount of material (target 140 and non-target 150) that bonds to the probe, thereby resulting in the energy-emitting signal at the corresponding location. It is understood that the MM probe 120 repels the target sequence 140, whereas the PM probe 120 binds to the target material 140 an amount S. Moreover, nonspecific sequences and other noise fragments can create significant interference if they are present in a significant concentration; thus, some amount N of non-target material 150 bonds to each probes 120, 130. (As used herein, “noise” comprises a probe's signal component that is not attributable to the bonding of the target material, including the cross-hybridization of ambient genes to the probe as well as other background effects.)

Accordingly, the existing technique relies on the MM probe 130 to provide a measure of the bonding of non-target material 150 to the PM probe 120—i.e., the binding of sequence species in the hybridization fluid having non-specific or partially-specific homology to the correct sequence. The amount of binding of non-target material 150 to each probe 120, 130 is defined as N. The traditional approach thus assumes the measured signal of the PM probe 120 is (S+N), whereas the measured signal of the MM probe 130 is N. Accordingly, under this approach, the “true” expression of the target material 140 is determined by subtracting the MM signal from the PM signal, thereby removing the noise, N, from the “true” target expression, S.

But this approach fails to accurately extract the true gene expression from the noise in the assay, in part because it ignores the effect of cross-hybridization of the MM probe with the non-target material due to its high homology with the PM probe. FIG. 2 is a comparison plot of the logarithmic gene expressions corrected by subtracting the MM signal from the PM signal. A comparison plot is a plot of the expressions of each gene against itself, as measured in two single-channel assays or in one dual-channel assay. A single-channel assay produces one set of gene expressions, whereas a dual-channel assay produces two independent values for each gene expression (e.g., using two different phosphorescent, fluorescent, or other energy-emitting markers that produce distinctly readable colors). In a comparison plot where each channel represents the same experiment, the data points theoretically fall on a straight diagonal line from the origin (where y=x), since the expression levels should be the same. In reality, noise due for example to cross-hybridization disturbs the signals, causing the data points to deviate from this line.

As shown in the plot of FIG. 2, that data are relatively good in region R_{H}, where the genes have a relatively high expression, but not in region R_{L}, where the genes have a relatively low expression. Effectively, the signal to noise ratio of the data points (corresponding to probes) in this region is too low for the data to be useful. Accordingly, techniques for more accurately determining the expressions of target materials in a biological assay where measured signals are affected by nonspecific binding and other noise effects are needed.

### SUMMARY OF THE INVENTION

To address this need, a Signal Allocation Model (SIAM) more accurately models the biological phenomena in an assay, allowing the useful biological information to be extracted from the assay data, which includes noise. An embodiment of the SIAM relates the measured signals of a plurality of probes to the true expressions of corresponding target materials. The SIAM thus enables a researcher to analyze and correct the biological assay data, even where the expression of the target material is relatively low.

An embodiment of the SIAM uses the concept that the signal of any probe targeting a particular material comprises contributions from the targeted material, non-targeted materials (i.e., any materials other than the target material), and possibly other background effects. Moreover, the contribution of each material to a probe signal varies with the biochemical affinity of the material to the probe. Accordingly, the SIAM first allocates the true target material expression and noise effects (e.g., the expressions of non-target materials) to each of the probes' measured signals. In one aspect, the allocations are based on the affinity of the target material to each probe, which in one embodiment is determined by the homologies between the material and the probe. Then, using the data obtained from the assay and based on these allocations, the corrected expression values are obtained according to the SIAM to obtain more accurate results for the true expression of the target material. In another embodiment, multiple probes correspond to different materials, and the SIAM is used to determine the expressions for each material.

In one embodiment, a microarray includes at least one probe-pair that comprises perfect match and mismatch probes. The perfect match probe corresponds to a target material, whereas the mismatch probe has a perturbation relative to the perfect match probe. The SIAM allocates the expressions of a target material and non-target material to the signals of the perfect match and mismatch probes. Under the SIAM approach, therefore, the signal from each probe (perfect match and mismatch) is explained by contributions from target and non-target material. Based upon the allocations and the measured signals, the expression of the target material (and possibly the noise effect) is determined.

In one embodiment, the target material is a gene, or a particular subsequence of a gene, wherein the assay includes a corresponding target probe and at least one homologous probe. The SIAM allocates the true gene expressions to each probe based on each gene's homology to the probe, where the homology is determined based on the genetic sequences of the probe and the genes.

In another aspect of the invention, a computer program product or a programmed computer system implements one or more of the functionalities described above. Another aspect of the invention is a set of assay data corrected according to the methods described herein, the data stored on a computer readable medium.

### BRIEF DESCRIPTION OF THE DRAWINGS

FIG. 1 is a side-view diagram of an existing model for an assay in which material binds to a probe-pair on a microarray, the probe-pair including a perfect match probe and a mismatch probe.

FIG. 2 is a comparison plot of data obtained from the assay of FIG. 1.

FIG. 3 is a side-view diagram of an embodiment of the SIAM for an assay using a microarray having probe-pairs that include perfect match and mismatch probes.

FIG. 4 is a graph of assay data for empirically determining the coefficients f_{S} and f_{N} according to one embodiment.

FIG. 5 is a comparison plot of corrected assay data in accordance with an embodiment of the invention.

FIG. 6 is a diagram of a computer-enabled system for performing an embodiment of the SIAM.

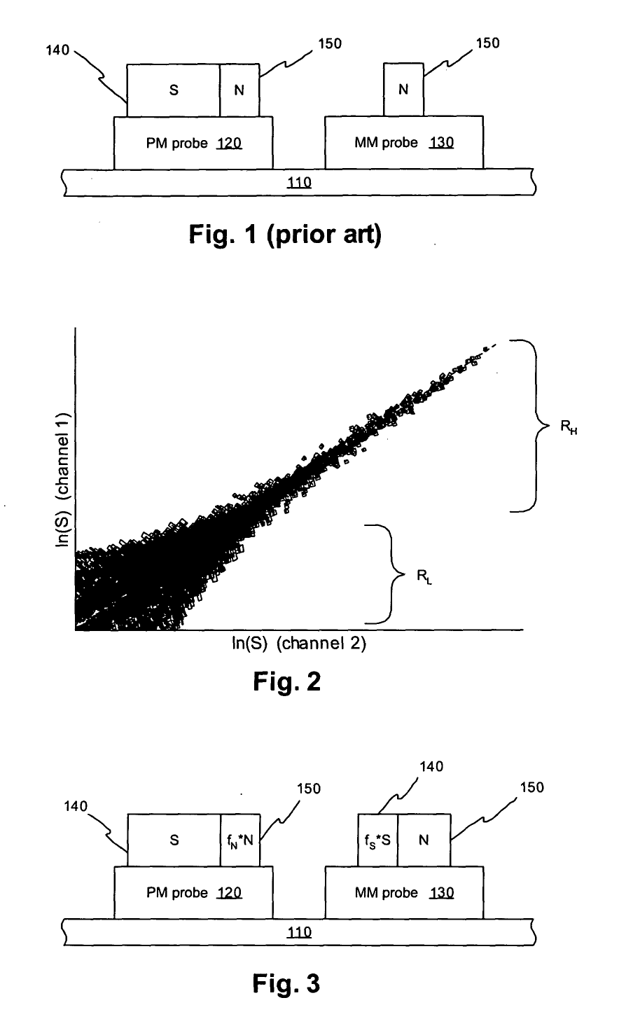

### DETAILED DESCRIPTION OF THE PREFERRED EMBODIMENTS

SIAM for PM-MM Probe-Pairs

Because of cross-hybridization, an oligonucleotide substrand or subsequence of a truly expressed gene will tend to bind to its perfect match probe on the array, but it will also bind at the mismatch location to a lesser but still significant extent. In addition, the impact of noise fragments on the mismatch probe is expected to be slightly greater than the impact on the perfect match location. This is further complicated by the scope and distribution of possible sequences, ranging from non-specific to partially-specific to near-specific match to the gene subsequence. In the low-match region, the expected low-level noise activity is nearly the same for both member pairs since the binding is driven mainly by concentration, while toward the near-match zone, the nuisance sequences begin to behave more like real gene subsequences but noisier. Therefore, for a truly expressed gene and partially-matched noise subsequences, the mismatch signal can exceed the perfect match signal.

In one embodiment, a model based on these concepts effectively deconvolves and estimates the true gene signal separate from the true noise signal. The SIAM for a single PM-MM probe-pair allocates the expression of the target sequence and the expression of ambient genes and other noise effects to each of the PM and MM signals. This model includes a pair of linear deconvolution equations independent of the oligo-clone sequence:
PM=S+f_{N}N
MM=f_{S}S+N
where PM is the signal measured from the perfect match probe, MM is the signal measured from the mismatch probe, S is the signal due to expression of the targeted gene sequence, N is the noise signal, f_{N} is the fraction of noise binding to the perfect match probe, and f_{S} is the fraction of the targeted sequence binding to the mismatch (i.e., cross-hybridization). This deconvolution solution of the model yields:

$$S = \frac{PM - {f}_{N} MM}{1 - {f}_{S} {f}_{N}} .$$

Because the PM probe is designed to exactly match to a target, and the MM probe is designed to have a perturbation relative to the perfect match, it is expected that the PM signal will be greater than or equal to the MM signal. In some cases, however, the measured MM signal may be larger than the PM signal. This may be due to, for example, the presence of an undiscovered subsequence or gene in the sample that coincidentally matches to the MM probe. In one embodiment, this is dealt with by first solving for the expression of this unknown subsequence, S_{unknown}, by switching the MM and PM signals in the SIAM described above:
MM=S_{unknown}+f_{N}N′
PM=f_{S}S_{unknown}+N′
After determining the expression of the unknown, the PM and MM signals are corrected, for example, by subtracting out the modeled effect of the unknown subsequence:
PM_{corrected}=PM−f_{S}S_{unknown}
MM_{corrected}=MM−S_{unknown}
After the effect of the unknown subsequence is determined and removed, the corrected PM and MM signals are used in the SIAM to determine the “true” expression of the targeted gene:
PM_{corrected}=S_{target}+f_{N}N
MM_{corrected}=f_{S}S_{target}+N
The expression of the target sequence, S_{target}, and the noise are then determined according to the above equations.

FIG. 3 illustrates a schematic side view of a portion of a microarray in accordance with this embodiment of the SIAM. This model explains how both the PM and MM signals can be attributed to the binding of both target material 140 and non-target material 150 at each probe 120, 130. It can be appreciated that this model more accurately models the phenomenon because it accounts for, e.g., he effect of cross-hybridization on the MM probe 130 due to its high homology with respect to the PM probe. It further accounts for the expected reduction in noise at the PM probe 120 due to competition with the targeted sequence.

In one embodiment, the homology between two sequences is defined as the percentage of nucleotides that are the same in each. This definition is typically more useful for shorter sequences. In another embodiment, useful for longer genetic sequences, the homology between two sequences is defined according to the Blast E-value. In yet another embodiment, homology can be thought of broadly as a measure of the biochemical affinity between two materials (e.g., a target and a probe).

To obtain corrected gene expression using this embodiment of the SIAM, signals for the PM and MM probes are obtained from at least one probe-pair. The gene expression and noise effects are allocated to each of the PM and MM probe signals according to the SIAM, above, where these allocations in one embodiment are determined by the coefficients, f_{S} and f_{N}. Methods for determining these coefficients are described below. The gene expression, S, is then computed using solved SIAM equation.

It has been found that for one embodiment of a typical microarray, f_{S} is around 0.2 to 0.3 due to mismatched nucleic acid, and f_{N} is about 0.9 to 1.0 due to competition with the targeted sequence. The coefficient f_{S} effectively models the degree to which the targeted sequence binds to the MM probe. Accordingly, as the ratio PP/MM increases, the gene expression S is more “specific” to the PM probe, so the coefficient f_{S} should be smaller. Similarly, the coefficient f_{N} effectively models the degree to which the perfect match is affected by noise compared to the MM probe. The PM probe tends to repel noise towards the mismatch, which explains why f_{N} is typically slightly below unity.

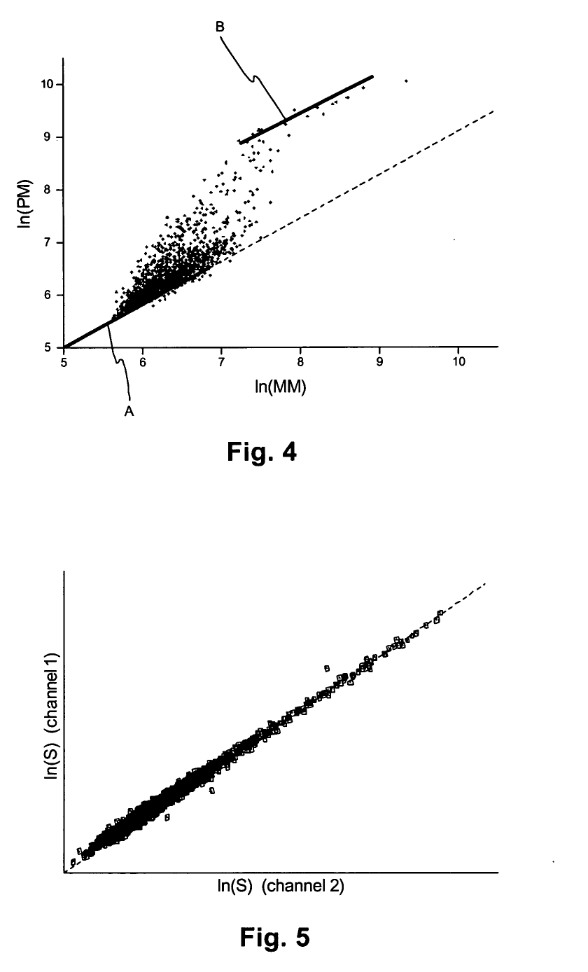

In another embodiment, the coefficients f_{S} and f_{N} are estimated using experimental and graphical methods. FIG. 4 is a graph of a single-channel assay of the PM signals versus the MM signals in an example microarray. The PM and MM signals are in logarithmic form for scaling purposes. It can be appreciated that, where the gene expression S for a particular probe-pair is relatively small (i.e., towards the bottom-left of the graph), the coefficient f_{N} can be approximated:
ln PM=ln MM≈ln f_{N}.
In addition, where the gene expression S for a particular probe-pair is relatively large (i.e., towards the upper-right of the graph), the coefficient f_{S} can be approximated:
ln MM−ln PM≈ln f_{S}.
For the example graphed in FIG. 4, the low-S approximation is useful near asymptote A, and the high-S approximation is useful for asymptote B. Using these approximations for this example data, it is determined that f_{N}≈0.09 and f_{S}≈0.33.

In addition to homology, the coefficients may depend on other variables, such as the total signal (PM+MM); the relative signal (PM/MM); and whether the sequence is a 5′ type sequence, 3′ type sequence, or middle type sequence. Although specific examples for determining the coefficients of the expression and noise values (and thus their allocations to each PM and MM signal) have been described, any of a number of techniques can be used. It is expected that the form and parameters for the coefficients will vary depending on the assay, the target materials, and several other experimental variables. For example, to determine the form and parameters of the coefficients for a particular assay, a researcher could perform an assay with a spiked sample or other verified biological sample. With the results of such an assay, the researcher would then attempt to fit the data in the model using different sets of coefficients. Varying the coefficients includes varying their functional form and parameters. Moreover, optimizing the coefficients can be performed globally across many arrays, and the resulting optimized global coefficients can be adapted or fine-tuned for each array.

The resulting corrected oligo-gene signal has much better precision than achieved by the conventional methods, as shown in the graph of FIG. 5. FIG. 5 is a graph of the corrected expression data from two single-channel arrays of identical biological samples. This comparison plot of corrected expression data from two single-channel arrays measuring the same biological sample produces the same pattern as a dual-channel, two-color array, which is inherently very precise for such comparisons. Notably, there is a significant improvement for low abundance genes, where noise previously rendered this data unusable.

Embodiments for the PM-MM probe-pair SIAM give a result for the expression of a particular target sequence. This determined expression of a target sequence provides an indication of the expression of a gene containing the target sequence. In another aspect of an embodiment, the gene expression is determined from the expressions of multiple different sequences associated with the gene, thereby improving the accuracy and reliability of the determined gene expression. In a typical assay using PM-MM probe-pairs, several probe-pairs are used to detect different subsequences of the same gene. Therefore, it is expected that the expressions of each of the target sequences correspond to the expression of the gene. Many techniques for computing the gene expression from a set of subsequence expressions are well known in the art, including a simple averaging the subsequence expressions and performing a linear regression on the SIAM model. In addition, more robust methods can be used to avoid “outliers,” including the median and the One-step Tukey Biweight Estimate. Determining the expression of a gene by targeting several subsequences generally produces more reliable results than determining a gene expression based on a single constituent subsequence.

Generalized SIAM

The SIAM can be applied more generally to any assay wherein a target material interacts with multiple homologous probes. For example, in an oligo-microarray, a probe's measured signal is due to the bonding of its corresponding target genetic sequence as well as contributions from ambient genes. Moreover, the contribution from each ambient gene varies with the biochemical affinity of the targeted and ambient genes to the various probe sequences. In the context of oligonucleotide bonding, the biochemical affinity between two oligonucleotide sequences is related to the homology between the sequences. These observations can be used to model and determine the actual expression signals for a set of genes based on the corresponding probes' measured signals and the homology between the sequences.

In one embodiment, an assay is conducted with a microarray that includes a number of probes comprising oligonucleotides immobilized at various locations on the microarray. The homology between any two probes can be determined if the sequences of each probe are known. Alternatively, the homology can be determined with well-known experimental techniques. A first probe is selected from the probes on a microarray, which is termed the target probe. It is assumed that homologous genes associated with other probes on the microarray also contribute to the target probe's signal, so these corresponding homologous probes are also selected. Accordingly, each probe in the set of selected probes has a homology relative to the target probe above a certain threshold level (e.g., 80%). However, there is no constraint on the homology between probes in the selected set, which may be below this threshold level.

Homology is a measure of the degree to which the probes will bind to the same target. The definition of homology can simply be the fraction of base components in a sequence that match the sequence of another, or it can take into account the locations of mismatch (e.g., a mismatch near the end of a sequences may reduce the homology of two sequences less than if the mismatch occurred in the middle of the sequences).

In one embodiment, the expressions of each of the materials are allocated to the target probe's signal and each of the set of selected homologous probes. Accordingly, the generalized SIAM can be described by the system of equations:

$$\begin{matrix}{T}_{1} = {f}_{11} {S}_{1} + {f}_{12} {S}_{2} + … + {f}_{1 M} {S}_{M} + {ε}_{1} \\ {T}_{2} = {f}_{21} {S}_{1} + {f}_{22} {S}_{2} + … + {f}_{2 M} {S}_{M} + {ε}_{2} \\ ⋮ \\ {T}_{M} = {f}_{M1} {S}_{1} + {f}_{M2} {S}_{2} + … + {f}_{M M} {S}_{M} + {ε}_{M}\end{matrix}$$

As with the embodiments described above, the coefficients fᵢⱼ effectively models the degree to which the j^{th} gene sequence bonds to the i^{th} probe. Accordingly, in one embodiment, the coefficients are determined by a monotonic function of the homology between the i^{th} and j^{th} sequences. In another embodiment, the coefficients are a function of the measured signal—e.g., the bonding of a material to its target probe is more specific for high expression levels, so the other coefficients (i≠Y) decrease as the signal levels increase. In one embodiment, the coefficients fᵢᵢ (i=j) are set to unity. Given the allocations as described in the system of equations above, the expressions, Sᵢ, are computed from the measured probe signals, Tᵢ. In an embodiment, a constraint (e.g., each expression is positive) is applied to the solution of the equations, which may give rise to the error terms εᵢ in the model. These error terms can be explained by the contributions of miscellaneous, non-modeled effects to the measured probe signals. 38

Once all of the coefficients and noise values are determined, the system of equations has an equal number of inputs (the measured probe signals, Tᵢ) as outputs (the gene expressions, Sᵢ). Therefore, the target gene expression prediction for T₁ can be determined by solving the system of equations using standard techniques, such as ordinary least squares. As a result, the corrected expression signals for each gene more accurately account for the effects of cross-hybridization between homologous sequences. The corrected data will resembled those shown in the plot of FIG. 5. As the plot shows, these data are more likely to be usable, for example, for low abundance genes having relatively low expression signals.

Because the probes were selected based on their homology relative to the target probe, it is expected that the model described above give the best results for the expression of the gene associated with the target probe. In part, this is because the probes in the selected set (i.e., where i=2, . . . , M) do not necessarily have a homology relative to every other probe that is higher than the predetermined threshold level (e.g., 80%). Therefore, the technique described above likely gives the best results for the target probe (T₁).

Accordingly, in another embodiment, the technique is repeated for every probe, Tᵢ, for which a corresponding gene expression, Sᵢ, is desired. For example, another probe T₂ is selected, and a set of homologous probes are determined. This set of probes typically is not the same as the set selected relative to T₁, so this model is optimized for T₂, and the results of this model for the gene expression S₂ would likely be different. By repeating this process for each probe on the microarray, a more accurate set of gene expressions can be determined.

System/Software Architecture and Data Flow

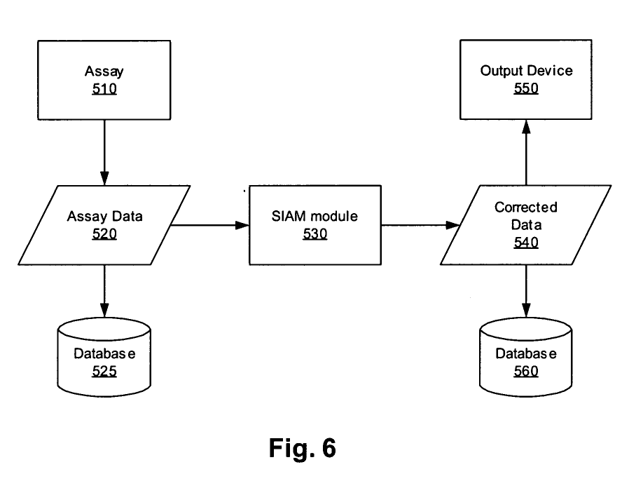

FIG. 6 illustrates an embodiment for performing the techniques described herein, for example on a computer system with appropriate computer software. It can be appreciated that any of the embodiments of the SIAM described above can be implemented with such a computer system or with any combination of well-known computational and data storage systems.

In one embodiment, a researcher conducts a biological assay 510, which results in a set of data 520. The assay data comprises a set of probe signals, which in one embodiment is the measured light intensities from the phosphorescence of each probe. Preferably, the assay data is stored in a database 525. The data are then received by a SIAM module 530, which is implemented by computer software running on a computer system. Preferably, the SIAM module 530 includes a means for reading the assay data in a standard format from the computer readable medium. In another embodiment, the SIAM module 530 is communicatively coupled to the experimental equipment used to perform the assay, such as a microarray adapted to produce computer readable signals from the experimental results. Alternatively, the researcher may manually input the assay data into the SIAM module 530, e.g., by using a computer keyboard or other input device.

The SIAM module 530 is programmed to correct the provided assay data using any of the embodiments of the SIAM herein described. This corrected data 540 is then provided to an output device 550, such as a display screen or printer, and/or to a database 560 or other computer-readable medium for electronic storage.

One benefit of the invention is that there is no requirement that the data be recently acquired. The SIAM may be used to correct “old” data that has been previously collected but could not be used because past techniques could not effectively extract the true expressions from the raw data. For example, a set of data like those shown in FIG. 2 could be corrected according to an embodiment of the SIAM to produce a set of corrected data like those shown in FIG. 5. In such a case, low gene expression data where the signal to noise ratio was previously too low is corrected with this system. Accordingly, the invention can be used to correct any biological assay data, regardless of when the assay was performed.

The foregoing description of the embodiments of the invention has been presented for the purposes of illustration and description. It is not intended to be exhaustive or to limit the invention to the precise forms disclosed. Persons skilled in the relevant art can appreciate that many modifications and variations are possible in light of the above teaching. It is therefore intended that the scope of the invention be limited not by this detailed description, but rather by the claims appended hereto.

1. A computer-implemented method for determining an expression of a target material in a biological sample given a set of measured signals from a set of probes, the set of probes including a target probe and one or more probes homologous to the target probe, the method comprising:
allocating the expression of the target material to each of the measured probe signals;
allocating the expressions of each of a set of non-target materials to each of the measured probe signals; and
based on the allocations, determining the expression of the target material.

2. The method of claim 1, wherein each of the homologous probes has a homology with the target probe higher than a threshold homology.

3. The method of claim 2, wherein the threshold homology is about 80%.

4. The method of claim 1, wherein the target material comprises an oligonucleotide and the probes comprise oligonucleotides immobilized on a microarray.

5. The method of claim 1, wherein said allocating comprises modeling each measured signal as a linear combination of a portion of the expression s of the target and non-target materials.

6. The method of claim 5, wherein each portion of a material's expression contributing to a probe's signal is a function of the homology between the material and the probe.

7. A computer-implemented method for determining an expression of a plurality of materials in a biological sample, given a set of measured signals from each of a set of probes, the method comprising:
selecting a target probe from the set of probes, the target probe associated with a target material;
allocating the expression of the target material and the expressions of non-target materials to each of a plurality of measured probe signals;
based on the allocations, determining the expression of the target material; and
repeating the allocating and determining steps with a different target probe selected from the set of probes.

8. The method of claim 7, wherein the target material comprises an oligonucleotide and the probes comprise oligonucleotides immobilized on a microarray.

9. The method of claim 7, wherein said allocating comprises modeling each measured signal as a linear combination of a portion of the expressions of the target and non-target materials.

10. The method of claim 9, wherein each portion of a material's expression contributing to a probe's signal is a function of the homology between the material and the probe.

11. A computer-implemented method for determining an expression of a nucleotide sequence in a biological sample, the biological sample having been put in contact with a probe-pair comprising a perfect match probe matching a subsequence of the nucleotide sequence and a mismatch probe having a perturbation relative to the perfect match probe, the method comprising:
allocating the nucleotide sequence expression and a fraction of a noise expression as components of a signal from the perfect match probe;
allocating the noise expression and a fraction of the nucleotide sequence expression as components of a signal from the mismatch probe; and
based on these allocations, determining the nucleotide sequence expression.

12. The method of claim 11, wherein the fraction of the nucleotide sequence expression allocated to the mismatch probe's signal is about 20% to about 30%.

13. The method of claim 11, wherein the fraction of the noise expression allocated to the perfect match probe's signal is about 90% to about 100%.

14. The method of claim 11, wherein the fraction of the nucleotide sequence expression allocated to the mismatch probe's signal and the fraction of the noise expression allocated to the perfect match probe's signal are determined empirically.

15. The method of claim 11, wherein the nucleotide sequence expression, S, is determined by the equation:
$$S = \frac{PM - {f}_{N} MM}{1 - {f}_{S} {f}_{N}} ,$$
where PM is the signal from the perfect match probe, MM is the signal from the mismatch probe, f_{N} is the fraction of the noise expression allocated to the perfect match probe's signal, and f_{S} is the fraction of the nucleotide sequence expression allocated to the mismatch probe's signal.

16. A computer program product having a computer readable medium, the computer readable medium having computer instructions encoded thereon for determining an expression of a target material in a biological sample given a set of measured signals from a set of probes, the set of probes including a target probe and one or more probes homologous to the target probe, the computer instructions comprising instructions for:
allocating the expression of the target material to each of the measured probe signals;
allocating the expressions of each of a set of non-target materials to each of the measured probe signals; and
based on the allocations, determining the expression of the target material.

17. The computer program product of claim 16, wherein the target material comprises a nucleotide sequence and the probes comprise nucleotide sequences immobilized on a microarray.

18. The computer program product of claim 16, wherein said allocating comprises modeling each measured signal as a linear combination of a portion of the expressions of the target and non-target materials.

19. The computer program product of claim 16, wherein each portion of a material's expression contributing to a probe's signal is a function of the homology between the material and the probe.

20. The computer program product of claim 16, wherein the computer instructions further comprise instructions for:
repeating the allocating and determining steps with a different target probe selected from the set of probes.

21. A computer program product having a computer readable medium, the computer readable medium having computer instructions encoded thereon for determining an expression of a nucleotide sequence in a biological sample, the biological sample having been put in contact with a probe-pair comprising a perfect match probe matching a subsequence of the nucleotide sequence and a mismatch probe having a perturbation relative to the perfect match probe, the computer instructions comprising instructions for:
allocating the nucleotide sequence expression and a fraction of a noise expression as components of a signal from the perfect match probe;
allocating the noise expression and a fraction of the nucleotide sequence expression as components of a signal from the mismatch probe; and
based on these allocations, determining the nucleotide sequence expression.

22. The computer program product of claim 21, wherein the fraction of the nucleotide sequence expression allocated to the mismatch probe's signal is about 20% to about 30%, and the fraction of the noise expression allocated to the perfect match probe's signal is about 90% to about 100%.

23. The computer program product of claim 21, wherein the nucleotide sequence expression, S, is determined by the equation:
$$S = \frac{PM - {f}_{N} MM}{1 - {f}_{S} {f}_{N}} ,$$
where PM is the signal from the perfect match probe, MM is the signal from the mismatch probe, f_{N} is the fraction of the noise expression allocated to the perfect match probe's signal, and f_{S} is the fraction of the nucleotide sequence expression allocated to the mismatch probe's signal.

24. A method comprising forwarding a result obtained from the method of claim 1, to a remote location.

25. A method comprising transmitting data representing a result obtained from the method of claim 1 to a remote location.

26. A method comprising receiving a result obtained from a method of claim 1 from a remote location.
